(19) United States
(12) Patent Application Publication
(10) Pub. No.: US 2016/0317080 A1
(43) Pub. Date: Nov. 3, 2016

(54) VEIN IMAGING ILLUMINATOR

(71) Applicant: Sensors Unlimited, Inc., Princeton, NJ (US)

(72) Inventors: Christopher B. Smith, West Windsor, NJ (US); Marc Hansen, Hamilton, NJ (US)

(73) Assignee: SENSORS UNLIMITED, INC., Princeton, NJ (US)

(21) Appl. No.: 14/699,561

(22) Filed: Apr. 29, 2015

(51) Int. Cl. A61B 5/00

(52) U.S. Cl. CPC A61B 5/489; A61B 5/0077; A61B 5/742; A61B 2560/0431

(57) ABSTRACT

A system for locating a patient's veins includes an illuminator configured to emit a conical beam for illuminating a target area of the patient and contrast between veins and surrounding tissue. The illuminator can be a short-wave infrared (SWIR) laser with a lens optic coupled to the SWIR laser for shaping the beam into a conical profile. The illuminator can be a visible light source with a reflective optic coupled to a filter configured to allow SWIR wavelengths therethrough.

### BACKGROUND OF THE INVENTION

1. Field of the Invention

The present invention relates to medical imaging of veins, and, in particular, to systems for imaging a patient's veins.

2. Description of Related Art

In the administration of medical care in an emergency situation, such as that encountered by a physician or emergency medical technician (EMT) in the treatment of an accident victim at the scene of the accident, or by a medic in the treatment of the wounded on a battlefield, the conditions under which the care is administered may be adverse, such as nighttime lighting conditions. It is well settled that expeditious administration of medical care to the victim improves the prospects of recovery. For example, the life of a wounded soldier on the battlefield may depend on the immediate intravenous administration of blood plasma or other lost body fluids or of medications. Similar immediate procedures by a physician or EMT may be required in order to treat a victim at the scene of an accident. Further, during transport of the victim to a hospital or similar medical care facility, administration of medical procedures may be necessary under low lighting or poor lighting conditions. Additionally, shock may have caused the veins of the victim to partially collapse, or the patient may have veins which are difficult to find which further complicates procedures for gaining access to the veins or arteries of the patient. Therefore, a need exists for technology to view the patient's veins accurately and quickly under adverse conditions. The present disclosure provides a solution for this need.

Such conventional methods and systems have generally been considered satisfactory for their intended purpose. However, there is still a need in the art for improved vein imaging systems.

### SUMMARY OF THE INVENTION

A system for locating a patient's veins includes an illuminator configured to emit a conical beam for illuminating a target area of the patient to contrast between veins and surrounding tissue.

The illuminator can include a short-wave infrared (SWIR) laser with a lens optic coupled to the SWIR laser for shaping the beam into a conical profile. The SWIR laser can be tuned to illuminate at a wavelength in the range of 0.9 μm and 1.7 μm. It is also contemplated that the illuminator can be a light source with a reflector coupled to a filter configured to allow SWIR wavelengths therethrough.

The illuminator can illuminate the target area with a substantially uniform beam. The conical beam can be angled 50° wide. The illuminator can be a handheld, portable device.

A method for locating a patient's veins includes illuminating a target area of the patient with a conical beam using an illuminator and receiving reflected illumination from the target area at an illumination sensor. Contrast between veins and surrounding tissue can be displayed through an imaging system.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1 is perspective view of an exemplary embodiment of the vein imaging illuminator constructed in accordance with the present disclosure, showing the illuminator in use, for example, on the battlefield to treat a wounded soldier.

FIG. 2 is a perspective view of an exemplary embodiment of the illuminator of FIG. 1, showing a conical beam focused on a target area;

FIG. 3 is a detailed view of one embodiment to of the illuminator of FIG. 1, showing a SWIR laser with a beam spreading lens optic; and

FIG. 4 is a detailed view of another embodiment of the illuminator of FIG. 1, showing a light source and reflective optic with a SWIR filter.

### DETAILED DESCRIPTION OF THE PREFERRED EMBODIMENTS

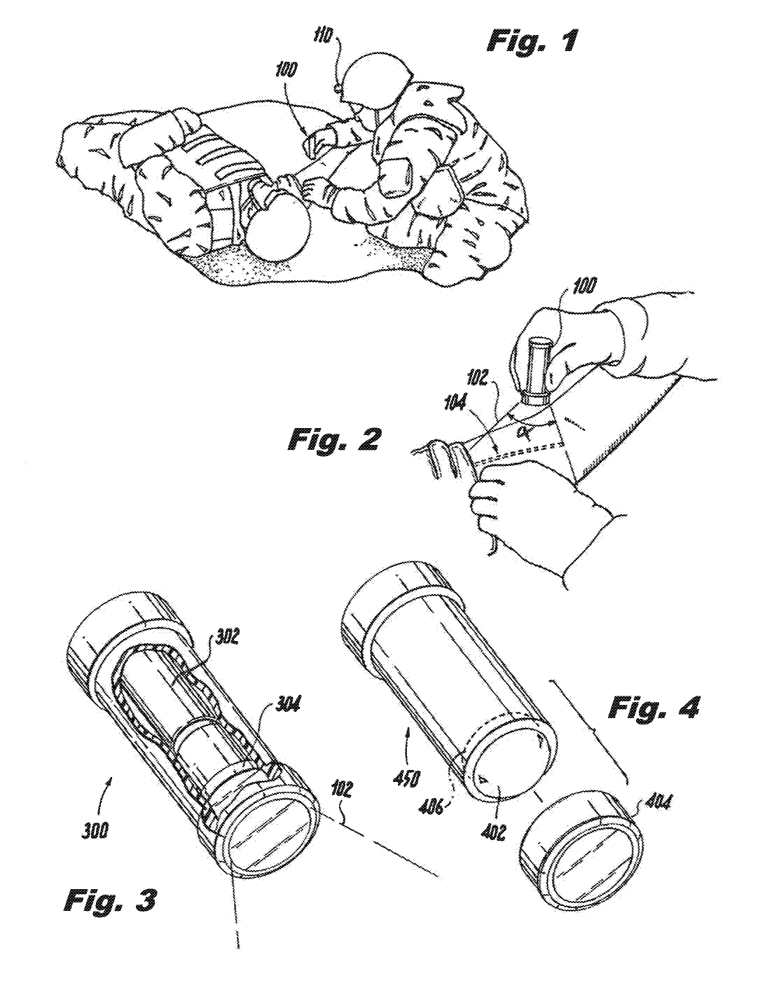

Reference will now be made to the drawings wherein like reference numerals identify similar structural features or aspects of the subject disclosure. For purposes of explanation and illustration, and not limitation, a partial view of an exemplary embodiment of a vein imaging illuminator in accordance with the disclosure is shown in FIG. 1 and is designated generally by reference character 100. Other embodiments of the illuminator in accordance with the disclosure, or aspects thereof, are provided in FIGS. 2-4, as will be described.

FIG. 1 illustrates the vein imaging illuminator 100 of the present disclosure in use, for example, on a battlefield to treat a wounded soldier. The illuminator 100 is portable, lightweight and can be battery operated so that the illuminator can be stored, carried and operable in remote locations. FIG. 2 shows a more detailed view of the illuminator 100 being used on a patient's arm. The illuminator 100 is configured to emit a conical beam 102 to illuminate a target area 104 of the patient. In doing so, the illuminator 100 provides a contrast between veins and surrounding tissue. This provides medical personnel facilitated access to the patient's veins even in low light, no light or adverse conditions.

With continued reference to FIG. 2, the best image from the illuminator 100 is obtained when the conical beam 102 has an angle α of 50° with the target area 104 and is substantially uniform along the target area 104. The illuminator 100 operates at short-wave infrared (SWIR) wavelengths that allow the conical beam 102 to be detected using SWIR imaging systems. An imaging system 110 such as a SWIR camera can display the veins contrasted from surrounding tissue to the medical personnel. The SWIR illuminator is not visible to the naked eye or to most night vision system. This is applicable, for example, to military personnel, when treating patients on a battlefield which would typically require the use of the artificial illumination that would be detectable and risk exposure to enemy identification. In the present disclosure the imaging system 110 is shown as a SWIR camera already installed on a helmet of the treating military personnel. The image from the camera can be viewed by the technician, e.g., on a goggle screen. However it will be understood that any imaging system capable of displaying SWIR images can be utilized with the illuminator 100 without departing from the scope of the present disclosure. For example, a SWIR imaging system can be stored in an ambulance or other emergency vehicle such that the treating emergency medical technician can view a patient's veins with improved contrast when responding to a nighttime accident.

SWIR illumination and imaging has been shown in conjunction with the discovery disclosed herein to considerably improve the contrast between veins and surrounding tissue compared to conventional visible illumination. The increased contrast in vein imaging using SWIR illumination can facilitate the administration of intravenous treatment in any suitable setting, include routine hospital treatments as well as in conditions with poor lighting, such as nighttime accidents, battlefields, or the like.

With reference to FIG. 3, a first embodiment 300 of the illuminator is shown. The illuminator 300 is a SWIR laser 302 with a lens optic 304 coupled to the SWIR laser for shaping the beam into the conical profile 102. In this embodiment, the SWIR laser wavelength ranges from 0.9 μm to 1.7 μm with an exemplary wavelength at 1.064 μm.

With referenced to FIG. 4, a second embodiment of the illuminator 400 is shown. The illuminator 400 is a visible light source 402 and a reflective optic 406 for attaining the conical beam 102. A SWIR filter 404 is coupled to the light source 402. The SWIR filter 404 is configured to allow a narrow bandwidth optimized to maximize SWIR imaging contrast between veins and surrounding tissue. In this embodiment, the SWIR filter 404 allows wavelengths in the range of from 0.9 μm to 1.7 μm to pass through.

The methods and systems of the present disclosure, as described above and shown in the drawings, provide for a vein imaging illuminator with superior properties including isolating a patient's veins in low light or no light conditions. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A system for locating a patient's veins, comprising:
an illuminator configured to emit a conical beam for illuminating a target area of the patient to contrast between veins and surrounding tissue.

2. The system of claim 1, wherein the illuminator is a short-wave infrared (SWIR) laser.

3. The system of claim 2, further comprising a lens optic coupled to the SWIR laser for shaping the beam into a conical profile.

4. The system of claim 2, wherein the SWIR laser is tuned to illuminate wavelength at a range from 0.9 μm and 1.7 μm.

5. The system of claim 1, wherein the illuminator is a visible light source with a reflective optic.

6. The system of claim 5, further comprising a filter coupled to the light source configured to allow SWIR wavelengths therethrough.

7. The system of claim 1, wherein the light source illuminates the target area with a substantially uniform beam.

8. The system of claim 1, wherein the conical beam is angled 50° with the target area.

9. The system of claim 1, wherein the illuminator is a handheld, portable device.

10. A method for locating a patient's veins, the steps:
illuminating a target area of the patient with a conical beam using an illuminator;
receiving reflected illumination from the target area at an illumination sensor; and
displaying a contrast between veins and surrounding tissue through an imaging system.

11. The method of claim 10, further comprising illuminating the target area with a SWIR laser.

12. The method of claim 11, further comprising transmitting the laser beam through a lens optic to generate the conical beam profile.

13. The method of claim 10, further comprising illuminating the target area with a visible light source and reflective optic for attaining the conical beam.

14. The method of claim 13, further comprising transmitting the conical beam through a SWIR filter.
